SEQUENCE LISTING (16 sequences)

SEQ ID NO: 1 - DNA, 22 bp, Artificial Sequence
  Forward primer for mycoplasma
atccaggtacgggtgaagacac

SEQ ID NO: 2 - DNA, 23 bp, Artificial Sequence
  Reverse primer for mycoplasma
cgcatcaacaagtcctagcgaac

SEQ ID NO: 3 - DNA, 20 bp, Artificial Sequence
  Forward primer for Escherichia coli
aggccttcgggttgtaaagt

SEQ ID NO: 4 - DNA, 20 bp, Artificial Sequence
  Reverse primer for Escherichia coli
gttagccggtgcttcttctg

SEQ ID NO: 5 - DNA, 15 bp, Artificial Sequence
  Fluorescently labeled probe for mycoplasma
  misc_feature (1)..(1): FAM
  misc_feature (15)..(15): NFQ-MGB
cgggacggaaagacc

SEQ ID NO: 6 - DNA, 15 bp, Artificial Sequence
  Fluorescently labeled probe for mycoplasma
  misc_feature (1)..(1): FAM
  misc_feature (15)..(15): BHQ1
cgggacggaaagacc

SEQ ID NO: 7 - DNA, 22 bp, Artificial Sequence
  Forward primer for pertussis
atccaggtacgggtgaagacac

SEQ ID NO: 8 - DNA, 23 bp, Artificial Sequence
  Reverse primer for pertussis
cgcatcaacaagtcctagcgaac

SEQ ID NO: 9 - DNA, 22 bp, Artificial Sequence
  Fluorescently labeled probe for pertussis
  misc_feature (1)..(1): FAM
  misc_feature (22)..(22): NFQ-MGB
aatggcaaggccgaacgcttca

SEQ ID NO: 10 - DNA, 22 bp, Artificial Sequence
  Fluorescently labeled probe for pertussis
  misc_feature (1)..(1): FAM
  misc_feature (2)..(2): BHQ1
aatggcaaggccgaacgcttca

SEQ ID NO: 11 - DNA, 29 bp, Artificial Sequence
  Forward primer for adenovirus
gacatgactttcgaggtcgatcccatgga

SEQ ID NO: 12 - DNA, 26 bp, Artificial Sequence
  Reverse primer for adenovirus
ccggctgagaagggtgtgcgcaggta

SEQ ID NO: 13 - DNA, 20 bp, Artificial Sequence
  Forward primer for Bacillus subtilis
tgaaggtgacgctgagtacg

SEQ ID NO: 14 - DNA, 20 bp, Artificial Sequence
  Reverse primer for Bacillus subtilis
tttttcagtgtcgcgttctg

SEQ ID NO: 15 - DNA, 20 bp, Artificial Sequence
  Fluorescently labeled probe for adenovirus
  misc_feature (1)..(1): FAM
  misc_feature (20)..(20): NFQ-MGB
gagtgcaccagccacaccgc

SEQ ID NO: 16 - DNA, 20 bp, Artificial Sequence
  Fluorescently labeled probe for adenovirus
  misc_feature (1)..(1): FAM
  misc_feature (20)..(20): BHQ1
gagtgcaccagccacaccgc

(19) United States
(12) Patent Application Publication
(10) Pub. No.: US 2018/0073069 A1
(43) Pub. Date: Mar. 15, 2018

(54) NUCLEIC ACID AMPLIFIER, CARTRIDGE FOR NUCLEIC ACID AMPLIFICATION AND NUCLEIC ACID AMPLIFICATION METHOD

(71) Applicant: SEIKO EPSON CORPORATION, Tokyo (JP)

(72) Inventor: Masayuki UEHARA, Gifu (JP)

(21) Appl. No.: 15/565,628

(22) PCT Filed: May 20, 2016
(86) PCT No.: PCT/JP2016/002466

§ 371 Date: Oct. 10, 2017

(30) Foreign Application Priority Data
May 26, 2015 (JP) 2015-106175

(51) Int. Cl. C12Q 1/68; C12M 1/34; C12P 19/34; C12N 15/10; B01L 3/00; B01L 7/00

(52) U.S. Cl. CPC C12Q 1/6858; C12M 1/34; C12Q 1/6806; C12Q 1/6818; B01L 7/52; C12P 19/34; C12N 15/1003; C12N 15/1065; B01L 3/5082; C12Q 1/6876

(57) ABSTRACT

A nucleic amplifier is provided to reduce an amplification efficiency drop even with a short cycle time. In the nucleic amplifier, the denaturation phase of moving and retaining a liquid droplet in a first region of a container heated to a denaturation temperature of a target nucleic acid, and the synthesis phase of moving and retaining the liquid droplet in a second region of the container different from the first region are repeated in multiple cycles. The liquid droplet contains a fluorescently labeled probe. The fluorescently labeled probe contains a minor groove binder molecule.

### BACKGROUND

### Technical Field

The present invention relates to a nucleic acid amplifier, a cartridge for nucleic acid amplification, and a nucleic acid amplification method.

### Background Art

PCR (polymerase chain reaction) is a technique used to amplify nucleic acid in repeated cycles of temperature changes by taking advantage of the difference in the way a nucleic acid undergoes denaturation and annealing according to differences such as nucleic acid chain length. The number of copies of nucleic acid produced by PCR is two raised to the power of a given number of cycles.

Applicant of this patent application has proposed a nucleic acid amplifier based on PCR, as described in JP-A-2012-115208 disclosing a PCR apparatus. The PCR apparatus disclosed in JP-A-2012-115208 includes a biotip installed therein and having a channel provided to move a reaction solution containing a target nucleic acid and other components. The channel contains the reaction solution, and is charged with a liquid having a smaller specific gravity than the reaction solution and that is not miscible with the reaction solution.

The PCR apparatus disclosed in JP-A-2012-115208 includes a heating section that heats a first region of the channel formed in the biotip, and a heating section that heats a second region at a temperature different from the heating temperature of the first region upon the biotip being installed in a mount provided for installation of the biotip. The PCR apparatus of JP-A-2012-115208 also includes a drive mechanism that switches the positions of the mount and the heating section between a first position and a second position. With the drive mechanism, the reaction solution in the biotip installed in the mount is moved between the first and second regions that are heated to different temperatures. The PCR apparatus of JP-A-2012-115208 is intended to shorten the amplification reaction time as compared to when the temperature is switched for the whole biotip.

There are demands to more quickly produce PCR products in the PCR apparatus above. However, it is of concern that the amplification efficiency drops when the cycle time producing a PCR product is made too short.

It is accordingly an object of the present invention to reduce an amplification efficiency drop even with a short cycle time.

### SUMMARY

A possible cause of amplification efficiency drop is the failure of a probe to bind to the template nucleic acid even when primers have successfully bound to the sequence. To investigate, the present inventor conducted intensive studies from the perspective of making the binding of a probe stronger even with a short nucleic acid synthesis reaction time. The studies led to the nucleic acid amplifier, the cartridge for nucleic acid amplification, and the nucleic acid amplification method of the present invention.

A nucleic acid amplifier of the present invention includes: a mount for installing a container containing a liquid droplet and an oil, the liquid droplet containing a template nucleic acid and a reagent used for amplification of a target nucleic acid in the template nucleic acid, the oil having a specific gravity different from a specific gravity of the liquid droplet and being in a separate phase from the liquid droplet; a first heater that sets a first region of the container installed in the mount to a denaturation temperature of the target nucleic acid, and a second heater that sets a second region of the container different from the first region to a synthesis temperature of the target nucleic acid; a moving mechanism that moves the liquid droplet from the first region to the second region, and from the second region to the first region; and a control unit that controls the moving mechanism so as to repeat a denaturation phase of retaining the liquid droplet in the first region, and a synthesis phase of retaining the liquid droplet in the second region in multiple cycles, the reagent containing a DNA polymerase, primers, a dNTP, and a fluorescently labeled probe, the fluorescently labeled probe containing a minor groove binder molecule.

The present invention includes: a container to which a liquid droplet of a solution containing a template nucleic acid is introduced, and that has a channel for moving the liquid droplet; and a reagent contained in the container, and that is used for amplification of a target nucleic acid in the template nucleic acid, the reagent containing a DNA polymerase, primers, a dNTP, and a fluorescently labeled probe, the fluorescently labeled probe containing a minor groove binder molecule.

A nucleic acid amplification method of the present invention includes: a temperature adjusting step of setting a target nucleic acid denaturation temperature for a first region of a container containing a liquid droplet containing a template nucleic acid and a reagent used for amplification of a target nucleic acid in the template nucleic acid, and setting a target nucleic acid synthesis temperature for a second region different from the first region; and an amplification step of repeating a denaturation phase and a synthesis phase in multiple cycles, the denaturation phase being a phase in which the liquid droplet is moved and retained in the first region, and the synthesis phase being a phase in which the liquid droplet is moved and retained in the second region, the reagent containing a DNA polymerase, primers, a dNTP, and a fluorescently labeled probe, the fluorescently labeled probe containing a minor groove binder molecule.

### BRIEF DESCRIPTION OF DRAWINGS

FIG. 1 is a diagram illustrating a cross section of a cartridge.

FIG. 2 is a diagram schematically representing a structure of a fluorescently labeled probe.

FIG. 3 is a diagram illustrating how a template nucleic acid solution is introduced into a container of the cartridge.

FIG. 4 is a diagram illustrating a state after a freeze dried reagent has returned to the original state in the presence of the template nucleic acid solution.

FIG. 5 is a block diagram of a nucleic acid amplifier.

FIG. 6 is a diagram representing an overview of a rotation mechanism.

FIG. 7 is a diagram showing a state after the cartridge is installed in a mount.

FIGS. 8A-8D are diagrams representing a thermal cycle process.

FIG. 9 is a flowchart representing the procedure of the thermal cycle process.

FIG. 10 is a graph showing amplification curves for Example 1 and Comparative Example 1.

FIG. 11 is a graph showing amplification curves for Example 2 and Comparative Example 2.

FIG. 12 is a graph showing amplification curves for Example 3 and Comparative Example 3.

### DETAILED DESCRIPTION

An exemplary embodiment of the present invention is described below with reference to the accompanying drawings. The exemplary embodiment and examples below are intended to help understand the present invention, and are not to be construed to limit the present invention. Various changes and modifications may be made to the present invention, provided that such changes remain within the gist of the present invention.

### (1) Embodiment

The following embodiment first describes the cartridge installed in a nucleic acid amplifier for amplifying nucleic acid, and the nucleic acid amplifier will be described later.

### Cartridge

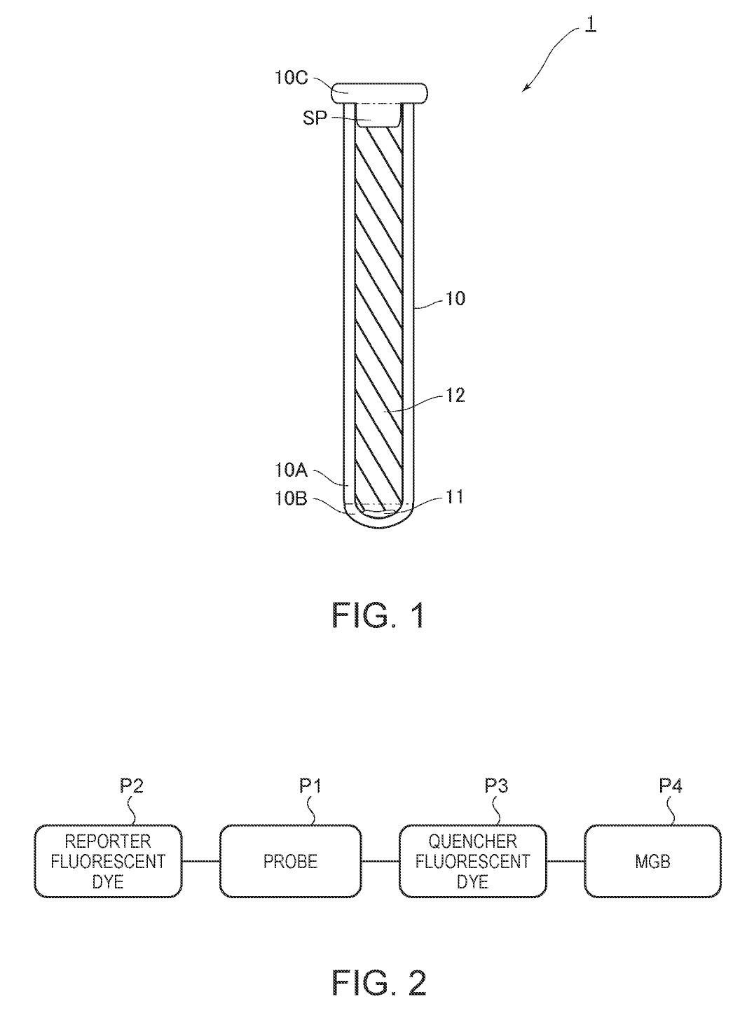

FIG. 1 is a diagram illustrating a cross section of a cartridge 1. As illustrated in FIG. 1, the cartridge 1 includes a container 10 for containing a reagent 11, and an oil 12. In the present embodiment, the container 10 has a cylindrical side wall portion 10A, and a hollow hemispherical bottom wall portion 10B.

The container 10 has an opening at the end opposite the bottom wall portion 10B, and a cap 10C is fitted to the opening. The cap 10C is a lid member that closes the opening of the container 10, and is detachably provided for the container 10 in the present embodiment. In the present embodiment, the cap 10C has a cylindrical sealing portion SP that is housed inside the container 10.

A template nucleic acid solution is introduced into the container 10. The cap 10C is removed from the container 10 when introducing the template nucleic acid solution into the container 10, and is fitted back to the container 10 once the solution is introduced into the container 10.

The template nucleic acid solution is introduced into the container 10 in the following manner, for example. Specifically, a specimen such as cells derived from living organisms such as humans and bacteria, or virus particles is collected using a collection instrument such as a cotton swab, and a template nucleic acid is extracted from the specimen using a known extraction technique. This is followed by purification of a template nucleic acid solution (solution containing the template nucleic acid) to achieve a predetermined concentration, using a known purification technique. The composition of the solution in the template nucleic acid solution is, for example, water (distilled water, sterile water), or a Tris-EDTA solution (TE).

The reagent 11 is a reagent used for amplification reaction of a target nucleic acid. The reagent 11 is contained in the container 10 in a freeze dry state. The reagent 11 used in the present embodiment is fixed at the bottom wall portion 10B of the container 10 by being freeze dried. The reagent 11 contains at least a DNA (deoxyribonucleic acid) polymerase, primers, a dNTP (deoxyribonucleotide triphosphate), a fluorescently labeled probe, and a buffer. Because the moisture in the reagent 11 is lost upon freeze drying the reagent 11, the magnesium, potassium, or other ions contained in the buffer are fixed at the bottom wall portion 10B of the container 10.

The target nucleic acid is a nucleic acid to be amplified, and represents all or part of the template nucleic acid in the template nucleic acid solution introduced into the container 10. For example, the target nucleic acid is a DNA fragment, a cDNA (complementary DNA) fragment, or a PNA (peptide nucleic acid).

The fluorescently labeled probe is a fluorescence-labeled substance used to quantify the amplification amount of the nucleic acid. As shown in FIG. 2, the fluorescently labeled probe of the present embodiment is constructed from a probe P1, a reporter fluorescent dye P2 added to the 5′ end of the probe P1, a quencher fluorescent dye P3 added to the 3′ end of the probe P1, and a minor groove binder (MGB) molecule P4 added to the quencher fluorescent dye P3.

When the template nucleic acid is RNA (ribonucleic acid), cDNA of the RNA is obtained by using a reverse transcriptase, and primers for reverse transcriptase by containing these components as the reagent 11 used for amplification reaction of the target nucleic acid.

The oil 12 has a smaller specific gravity than the template nucleic acid solution introduced into the container 10, and undergoes phase separation from the template nucleic acid solution. The oil 12 is, for example, a 2CS silicone oil, or a mineral oil.

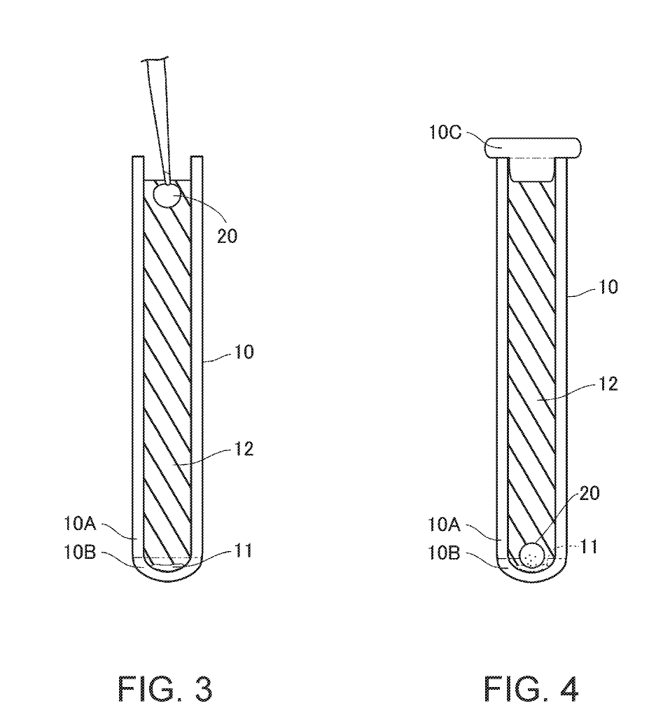

FIG. 3 is a diagram illustrating how the template nucleic acid solution is introduced into the container of the cartridge. FIG. 4 is a diagram illustrating a state after the freeze dried reagent has returned to the original state in the presence of the template nucleic acid solution.

As illustrated in FIG. 3, the template nucleic acid solution introduced into the container 10 of the cartridge 1 is acted upon by a force that reduces the surface area of the interface, and forms a liquid droplet 20 by undergoing phase separation from the oil 12 in the container 10. Because the liquid droplet 20 has a larger specific gravity than the oil 12, the liquid droplet 20 settles along the side wall portion 10A providing a channel for the liquid droplet 20 in the container 10. The liquid droplet 20 has a volume of preferably 0.2 μL to 2 μL.

As illustrated in FIG. 4, the freeze dried reagent 11 returns to the original state with the moisture of the liquid droplet 20 that has settled down to the bottom wall portion 10B of the container 10, and becomes incorporated in the liquid droplet 20.

With the reagent 11 incorporated in the liquid droplet 20 after returning to the original state from the freeze dried state, the liquid droplet 20 contains the template nucleic acid, and the reagent 11 used for amplification of the target nucleic acid in the template nucleic acid. That is, the liquid droplet 20 becomes a site of nucleic acid amplification reaction.

Preferably, the inner wall of the side wall portion 10A of the container 10 is water repellent to such an extent that there is no adhesion of the liquid droplet 20.

### Nucleic Acid Amplifier

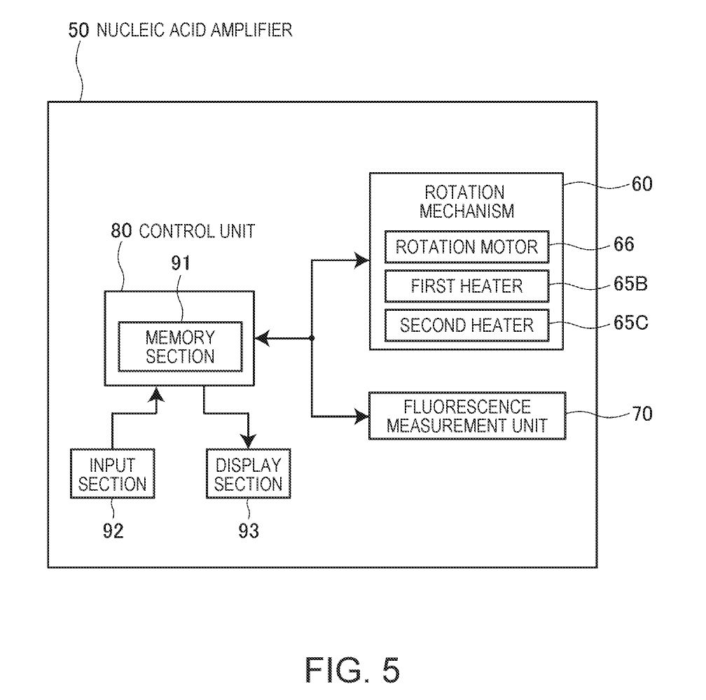

FIG. 5 is a block diagram of a nucleic acid amplifier. As shown in FIG. 5, a nucleic acid amplifier 50 includes a rotation mechanism 60, a fluorescence measurement unit 70, and a control unit 80.

### Rotation Mechanism

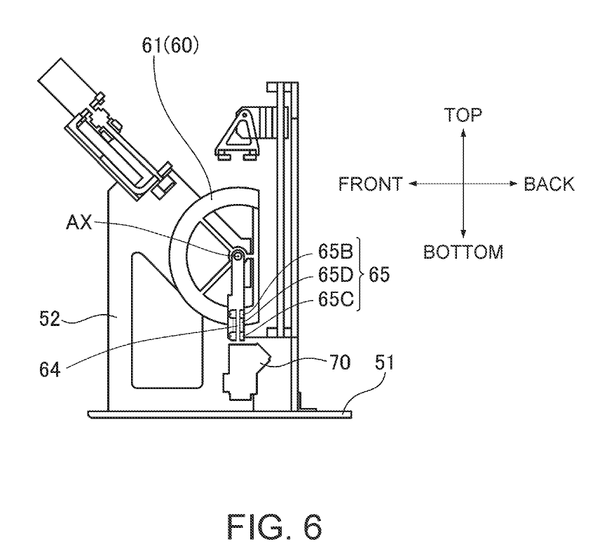

FIG. 6 is a diagram representing an overview of the rotation mechanism. FIG. 6 shows a side view of the rotation mechanism 60. In describing the nucleic acid amplifier 50, the vertical, depth, and lateral directions are defined as shown in FIG. 6. Specifically, the vertical direction is a direction in which the nucleic acid amplifier 50 stands upright with the base 51 of the nucleic acid amplifier 50 horizontally placed, and the terms “up” and “down” are used relative to the direction of gravity. The lateral direction is a direction along the rotational axis AX of the cartridge 1. The depth direction is a direction perpendicular to the vertical direction and the lateral direction.

As illustrated in FIG. 6, the rotation mechanism 60 has a rotor 61, and a rotation motor 66 for rotating the rotor 61 (FIG. 5). The rotor 61 is provided with a heating unit 65 having an insertion hole 64 that can accept and release the cartridge 1. The rotor 61 rotates about the rotational axis AX supported on a support 52 fixed to the base 51, without changing the relative positions of the heating unit 65, and the cartridge 1 installed in the insertion hole 64 of the heating unit 65.

In the present embodiment, the insertion hole 64 of the heating unit 65 serves both as a hole for inserting and removing the cartridge 1, and a mount for installing the cartridge 1 inserted into the hole. It is, however, possible to separately provide the hole and the mount in the nucleic acid amplifier 50. The number of cartridges 1 that can be installed in the mount is not limited to one, and more than one cartridge 1 may be installed in the amount.

Under the instruction of the control unit 80, the rotation motor 66 (FIG. 5) rotates the rotor 61 in such a manner that the cartridge 1 installed in the insertion hole 64 of the heating unit 65 is inverted upside down.

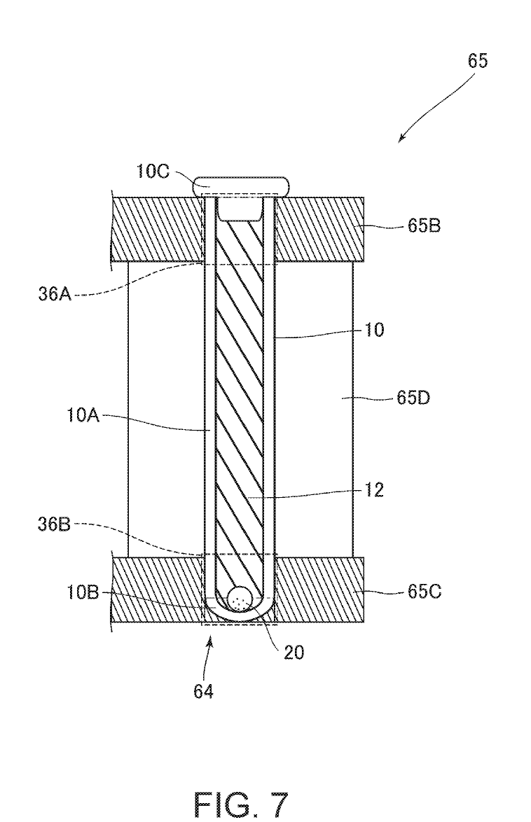

FIG. 7 is a diagram showing a state after the cartridge is installed. As illustrated in FIG. 7, the heating unit 65 includes a first heating unit 65B for applying heat at which the target nucleic acid undergoes a denaturation reaction, and a second heating unit 65C for applying heat at which the target nucleic acid undergoes a synthesis reaction (annealing reaction, and extension reaction).

With the cartridge 1 installed in the insertion hole 64 of the heating unit 65, the first region 36A at one end of the side wall portion 10A providing a channel for the liquid droplet 20 in the container 10 of the cartridge 10 becomes surrounded by the first heating unit 65B. The first heating unit 65B heats the first region 36A to, for example, 95 to 100° C.

With the cartridge 1 installed in the insertion hole 64 of the heating unit 65, the second region 36B at the other end of the side wall portion 10A providing a channel for the liquid droplet 20 in the container 10 of the cartridge 1 becomes surrounded by the second heating unit 65C. The second heating unit 65C heats the second region 36B to, for example, 50 to 75° C.

In this manner, the first region 36A of the container 10 in the cartridge 1 is heated to a temperature at which the target nucleic acid undergoes a denaturation reaction, and the second region 36B of the container 10 is heated to a temperature at which the target nucleic acid undergoes a synthesis reaction.

A spacer 65D for inhibiting conduction of heat between the first heating unit 65B and the second heating unit 65C is provided between these members. The spacer 65D has a through hole formed along the longitudinal direction of the insertion hole 64 through the first heating unit 65B and the second heating unit 65C so that the insertion of the container 10 of the cartridge 1 into the insertion hole 64 will not be obstructed.

### Fluorescence Measurement Unit

The fluorescence measurement unit 70 is a unit where the fluorescence intensity of the liquid droplet 20 contained in the container 10 of the cartridge 1 is measured. As shown in FIG. 6, the fluorescence measurement unit 70 is disposed opposite an end of the cartridge 1 installed in the insertion hole 64 of the heating unit 65, with a predetermined distance from the end of the cartridge 1.

Under the measurement instruction from the control unit 80, the fluorescence measurement unit 70 applies excitation light that corresponds to the fluorescent dye contained in the liquid droplet 20, and measures the emitted fluorescence intensity from the liquid droplet 20. The fluorescence measurement unit 70 sends the control unit 80 data obtained as the measurement result indicative of the measured fluorescence intensity. The fluorescence measurement unit 70 may be adapted to measure a fluorescence intensity corresponding to a single fluorescent dye, or fluorescence intensities corresponding to a plurality of fluorescent dyes.

### Control Unit

As illustrated in FIG. 5, the control unit 80 includes a memory section 91, and is connected to an input section 92, a display section 93, and other components. The memory section 91 includes a program storage region, a region for storing various data, including settings data sent from the input section 92, and data obtained after a nucleic acid amplification process, and a region where these programs and data are expanded.

The control unit 80 appropriately controls the rotation mechanism 60 and the fluorescence measurement unit 70 using the programs and the settings data stored in the memory section 91, and executes a thermal cycle process or an amplification analysis process, as appropriate.

### Thermal Cycle Process

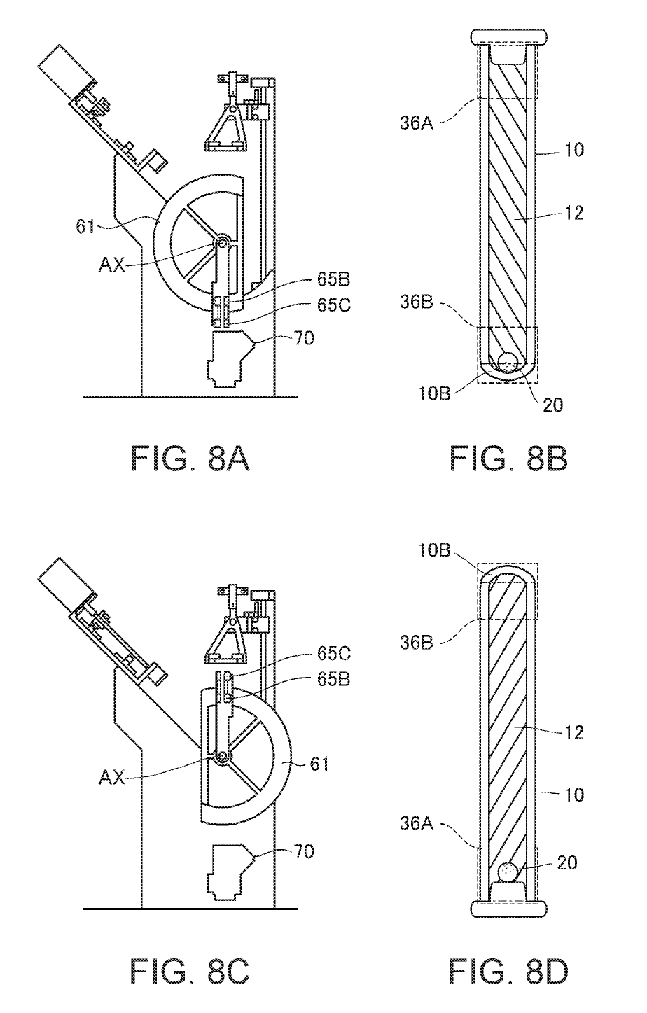

FIGS. 8(A)-(D) are diagrams representing a thermal cycle process. Specifically, FIG. 7 represents a target nucleic acid synthesis phase ((A) and (B)), and a target nucleic acid denaturation phase ((C) and (D)).

Specifically, upon receiving an instruction for starting, for example, a thermal cycle process from the input section 92, the control unit 80 drives the first heating unit 65B provided for the rotor 61, and heats the first region 36A of the container 10 in the cartridge 1 to a temperature at which the target nucleic acid undergoes a denaturation reaction. The control unit 80 also drives the second heating unit 65C provided for the rotor 61, and heats the second region 36B of the container 10 of the cartridge 1 to a temperature at which the target nucleic acid undergoes a synthesis reaction. This creates a temperature gradient in the oil 12 filling the container 10 of the cartridge 1.

It takes a predetermined time for the oil 12 to reach a temperature of, for example, 98° C. in the first region 36A, and to a temperature of, for example, 54° C. in the second region 36B after the driving of the first heating unit 65B and the second heating unit 65C. Because the target nucleic acid amplification reaction does not properly proceed during this time period, the control unit 80 is put to standby for the duration of this time period.

Here, as illustrated in FIG. 8(A), the rotor 61 is at a reference position where the cap 10C side of the container 10 installed in the insertion hole 64 of the heating unit 65 is on the upper side, and the bottom wall portion 10B side of the container 10 is on the lower side. With the rotor 61 at the reference position, the liquid droplet 20 settles down to the second region 36B under its weight, as shown in FIG. 8(B). Accordingly, the target nucleic acid contained in the liquid droplet 20 does not enter the first denaturation phase.

After the standby time, the control unit 80 rotates the rotor 61 180 degrees. Here, as shown in FIG. 8(C), the rotor 61 is in an inverted position where the cap 10C side of the container 10 installed in the insertion hole 64 of the heating unit 65 is on the lower side, and the bottom wall portion 10B side of the container 10 is on the upper side. With the rotor 61 in the inverted position, as shown in FIG. 8(D), the liquid droplet 20 settles down to the first region 36A under its weight. This causes the target nucleic acid contained in the liquid droplet 20 to enter the denaturation phase.

The control unit 80 keeps the rotor 61 stationary for a denaturation reaction period set for the target nucleic acid denaturation phase after the rotor 61 is rotated 180 degrees (after the rotation of the rotor 61 has stopped). This causes the denaturation reaction of the target nucleic acid contained in the liquid droplet 20 to proceed. The denaturation reaction period is at least the time it takes for the liquid droplet 20 to move between the first region 36A—a region at one end of the side wall portion 10A providing a channel for the liquid droplet 20 in the container 10, and the second region 36B at the other end of the side wall portion 10A. In the present embodiment, the denaturation reaction period is 2 seconds or more and less than 5 seconds, shorter than the commonly adopted denaturation reaction period of 5 seconds or more and less than 30 seconds.

After the denaturation reaction period has passed, the control unit 80 rotates the rotor 61 180 degrees to switch the rotor 61 position from the inverted position to the reference position, and move the liquid droplet 20 to the second region 36B, as shown in FIG. 8(B). This causes the target nucleic acid contained in the liquid droplet 20 to enter the synthesis phase.

The control unit 80 keeps the rotor 61 stationary for a synthesis reaction period set for the target nucleic acid synthesis phase after the rotor 61 is rotated 180 degrees (after the rotation of the rotor 61 has stopped). This causes the annealing and extension reaction of the target nucleic acid contained in the liquid droplet 20 to proceed. As with the case of the denaturation reaction period, the synthesis reaction period is at least the time it takes for the liquid droplet 20 to move between the first region 36A and the second region 36B. In the present embodiment, the synthesis reaction period is 3 seconds or more and less than 20 seconds, shorter than the commonly adopted denaturation reaction period of 20 seconds or more and less than 60 seconds.

In this manner, the control unit 80 alternately switches the inverted position and the reference position, and the denaturation phase of moving and retaining the liquid droplet 20 in the first region 36A, and the synthesis phase of moving and retaining the liquid droplet 20 in the second region 36B are repeated in multiple cycles. The number of cycles to be repeated is set in the control unit 80, and is, for example 50.

### Amplification Analysis Process

The amplification analysis process is performed simultaneously with the thermal cycle process. Specifically, the control unit 80 sends a measurement instruction to the fluorescence measurement unit 70 for each synthesis reaction period, and data indicative of the measured fluorescence intensity sent from the fluorescence measurement unit 70 is stored in the memory section 91 as a result of the measurement instruction.

As illustrated in FIGS. 8(A) and (B), the rotor 61 is at the reference position in the synthesis reaction period, and the liquid droplet 20 inside the container 10 settles toward the bottom wall portion 10B. However, there are cases where the liquid droplet 20 has not reached the bottom wall portion 10B immediately after the rotor 61 has moved to the reference position. It is therefore desirable that the control unit 80 send a measurement instruction to the fluorescence measurement unit 70 after a predetermine time period has passed from the completion of the rotation of the rotor 61 from the inverted position to the reference position, particularly, immediately before rotation from the reference position to the inverted position.

In response to an instruction from the input section 92, the control unit 80 reads from the memory section 91 data indicative of fluorescence intensity for the set number of repeated cycles, and, by using the read data, generates an amplification curve that indicates fluorescence intensity changes against the number of cycles. When generating an amplification curve, the control unit 80 determines how the results compare to the reference amplification efficiency using the amplification curve, and displays the result of determination, and/or the amplification curve on the display section 93, as appropriate.

### Procedure of Thermal Cycle Process

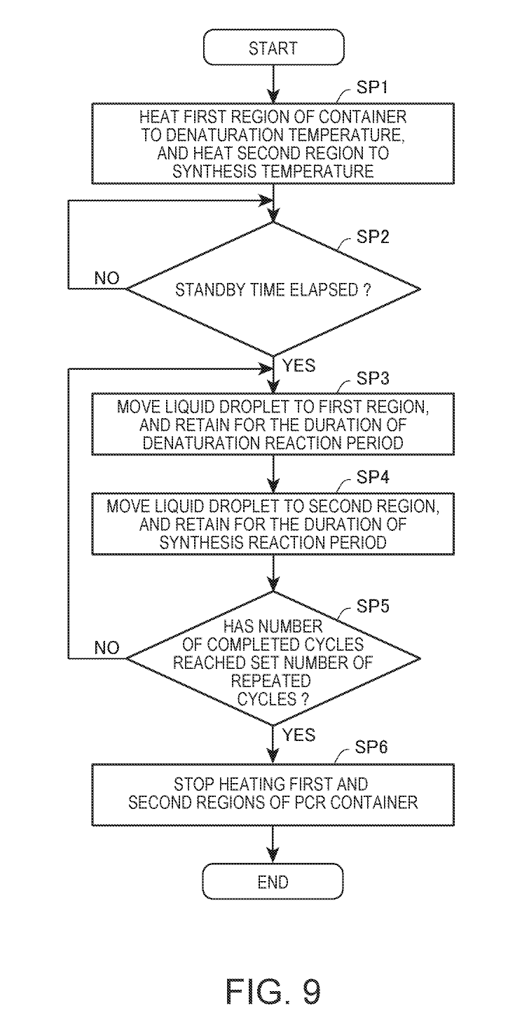

FIG. 9 is a flowchart representing the procedure of the thermal cycle process. As shown in FIG. 9, after a nucleic acid elution process, the control unit 80 proceeds to step SP1, and heats the first region 36A of the container 10 of the nucleic acid amplification cartridge 1 to the denaturation temperature that has been set as a temperature at which the target nucleic acid undergoes a denaturation reaction. The control unit 80 also heats the second region 36B of the container 10 to a synthesis temperature that has been set as a temperature at which the target nucleic acid undergoes a synthesis reaction. The sequence goes to step SP2 after step SP1.

In step SP2, the control unit 80 puts itself to standby until an elapse of a standby time—a time period from the start of heating to the heated subject reaching the target temperature. The sequence goes to step SP3 after the standby time has passed.

In step SP3, the control unit 80 rotates the rotor 61 from the reference position to the inverted position, and moves the liquid droplet 20 to the denaturation temperature region (first region 36A) of the container 10. The control unit 80 keeps the rotor 61 stationary until the denaturation reaction period elapses after the rotor 61 is moved to the inverted position, and retains the liquid droplet 20 in the denaturation temperature region of the container 10. After the denaturation reaction period has passed, the control unit 80 goes to step SP4.

In step SP4, the control unit 80 rotates the rotor 61 from the inverted position to the reference position, and moves the liquid droplet 20 to the synthesis temperature region (second region 36B) of the container 10. The control unit 80 keeps the rotor 61 stationary until the synthesis reaction period elapses after the rotor 61 is moved to the reference position, and retains the liquid droplet 20 in the synthesis temperature region of the container 10. After the synthesis reaction period has passed, the control unit 80 goes to step SP5.

In step SP5, the control unit 80 determines whether the number of completed cycles has reached the set number of cycles to be repeated. If the number of completed cycles has not reached the set number, the control unit 80 adds one cycle, and repeats the process from step SP3. The control unit 80 goes to step SP6 when the number of completed cycles has reached the set number.

In step SP6, the control unit 80 stops heating of the first region 36A and the second region 36B in the container 10, and ends the thermal cycle process.

### Brief Overview

As described above, in the cartridge 1 of the present embodiment, the reagent 11 used for amplification of the target nucleic acid in the template nucleic acid is contained in the container 10 in a freeze-dry state. The reagent 11 returns to the original state in the presence of the liquid droplet 20 of the template nucleic acid solution (a solution containing the template nucleic acid) introduced into the container 10, and becomes incorporated in the liquid droplet 20. In this way, the reagent 11 can be added without having a user make adjustments to the reagent 11 for the liquid droplet 20 that is introduced into the container 10. This makes it possible to save time needed to adjust the reagent 11, and avoid an amplification efficiency drop due to adjustment errors.

The liquid droplet 20 is moved by the nucleic acid amplifier 50. Specifically, the liquid droplet 20 is alternately moved to the first region 36A—a region heated to the denaturation temperature, and to the second region 36B—a region heated to the synthesis temperature, along the side wall portion 10A providing a channel in the container 10. In this way, the amplification reaction period (cycle time) can be shortened as compared to the common procedure in which the temperature is switched between denaturation temperature and synthesis temperature at a certain location. Specifically, when the liquid droplet 20 has a volume of 0.2 μL to 2 μL, the liquid droplet 20 can quickly move between the first region 36A and the second region 36B of the container 10, and the amplification reaction period (cycle time) can be made even shorter. A concern, however, is that a shorter synthesis reaction time may lead to poor amplification efficiency.

In the present embodiment, this is addressed by containing a DNA polymerase, primers, a dNTP, and a fluorescently labeled probe in the reagent 11. As illustrated in FIG. 2, the fluorescently labeled probe is constructed from a probe, a reporter fluorescent dye added to the 5′ end of the probe, a quencher fluorescent dye added to the 3′ end of the probe, and an MGB molecule added to the quencher fluorescent dye.

With the fluorescently labeled probe containing the MGB molecule, it was found that the amplification efficiency improves even when the synthesis reaction period is shorter than the commonly adopted synthesis reaction period of 20 seconds. This will be described later in Examples.

It is known that a fluorescently labeled probe containing an MGB molecule P4 maintains specificity even with a short probe length. A possible explanation for this is that the MGB molecule P4 approaches the corresponding minor groove of the template nucleic acid, and the probe P1 added to the MGB molecule P4 forms a specific complementary strand with the base sequence of the minor groove, and improves its binding efficiency or bonding strength.

Specifically, the MGB molecule P4 is a factor that is known to potentially maintain specificity even at low temperature. Our finding is that the MGB molecule P4 is also a factor that can maintain specificity even with a short synthesis reaction time. The finding adds a factor that can be adjusted to obtain a certain amplification efficiency, and widens the freedom of probe design, making it possible to improve both sensitivity and specificity for the target nucleic acid.

### (2) Variation

The embodiment uses the fluorescently labeled probe constructed from a probe P1, a reporter fluorescent dye P2 added to the 5′ end of the probe P1, a quencher fluorescent dye P3 added to the 3′ end of the probe P1, and a minor groove binder molecule P4 added to the quencher fluorescent dye P3. However, the minor groove binder molecule P4 may be added to the probe P1 or the reporter fluorescent dye P2.

A fluorescently labeled probe of an intercalation fluorescent dye may be used instead of the fluorescently labeled probe of the embodiment. It is also possible to use a fluorescent dye-binding fluorescently labeled probe, such as a cycling probe, instead of the fluorescently labeled probe of the embodiment. When using such a fluorescently labeled probe, the minor groove binder molecule P4 may be added to, for example, the fluorescent dye.

In short, the fluorescently labeled probe is not limited, as long as it contains the minor groove binder molecule P4.

In the embodiment, the side wall portion 10A of the container 10 is cylindrical, and the bottom wall portion 10B of the container 10 is a hollow hemispherical portion. However, the container 10 may have a variety of other shapes.

In the embodiment, the cap 10C is detachably provided for the container 10, and the template nucleic acid solution is introduced with the cap 10C removed from the container 10. However, the cap 10C may be fixed to the container 10, and the template nucleic acid solution may be introduced via a needle penetrating through the cap 10C. The cap 10C may be formed as an integral part of the container 10.

In the embodiment, the sealing portion SP of the cap 10C is cylindrical in shape. However, the sealing portion SP may have a hemispherical or a conical depression in a portion facing the bottom wall portion 10B of the container 10. When the depression is tapered away from the side wall portion 10A of the container 10, the liquid droplet 20 can be held static at a fixed position, and can be heated under more uniform conditions. The bottom wall portion 10B of the container 10 may be tapered away from the side wall portion 10A of the container 10.

In the embodiment, the liquid droplet 20 has a larger specific gravity than the oil 12. However, the liquid droplet 20 may have a smaller specific gravity than the oil 12. The effects described in the embodiment also can be obtained in this case.

In the embodiment, the denaturation reaction period and the synthesis reaction period start at the timing when the rotor 61 is fully rotated 180 degrees (when the rotor 61 stops). However, the start timing of the denaturation reaction period and the synthesis reaction period may be when the rotor 61 starts a 180-degree rotation.

In the embodiment, the rotation mechanism 60 is used as the mechanism that alternately moves the liquid droplet 20 inside the container 10 of the cartridge 1 between the first region 36A and the second region 36B. However, various other mechanisms other than the rotation mechanism 60 may be used, provided that the liquid droplet 20 is alternately moved between the first region 36A that is brought to the denaturation temperature of the target nucleic acid in the container 10, and the second region 36B different from the first region 36A and that is brought to the synthesis temperature of the target nucleic acid.

In the embodiment, the regions to which the liquid droplet 20 is moved inside the container 10 are the first region 36A that is brought to the denaturation temperature of the target nucleic acid, and the second region 36B different from the first region 36A and that is brought to the synthesis temperature of the target nucleic acid. However, for example, three regions may be disposed, as described in Japanese Patent Application No. 2014-107844. Specifically, the first region 36A is a region that is brought to the denaturation temperature of the target nucleic acid, and two different regions are disposed as the second region 36B. One of the second regions is brought to the annealing temperature that has been set as a temperature at which an annealing reaction takes place in the synthesis reaction of the target nucleic acid. The other second region is brought to the extension temperature that has been set as a temperature at which the target nucleic acid undergoes an extension reaction. Specifically, the invention is not limited to the embodiment in which the temperature changes in a cycle are a two-phase process including the denaturation phase and the synthesis phase. The liquid droplet 20 also can be moved inside the container in a cycle consisting of three phases of denaturation, annealing, and extension. A variety of moving mechanisms other than the rotation mechanism may be used also for a cycle of temperature changes occurring in three phases.

In the embodiment, the nucleic acid amplifier 50 is used that includes the first heating unit 65B and the second heating unit 65C. However, a nucleic acid amplifier different from the nucleic acid amplifier 50 of the embodiment may be used, provided that a temperature gradient can be created inside the container 10. For example, only a high-temperature heater may be provided, and the second heating unit 65C may be replaced with a cooler. It is also possible to use a high-temperature heater and a low-temperature heater provided outside of the rotor 61. As another example, the first heating unit 65B and the second heating unit 65C may be switched in position.

### EXAMPLES

### Example 1

First, a template nucleic acid, the reagent 11 used for amplification of a target nucleic acid in the template nucleic acid, and a positive control were charged into a test tube, and 10 μL of distilled water was added to prepare a template nucleic acid solution.

The mycoplasma pneumoniae genomic DNA (MBC035, 250 copies) available from Vircell was used as the template nucleic acid, and only 0.625 μL was charged into the test tube.

The Escherichia coli genomic DNA (9060, 750 fg/μL) available from Takara Bio was used as the positive control, and only 0.625 μL was charged into the test tube.

The Platinum Taq available from Life Technologies was used as the DNA polymerase contained in the reagent 11, and only 0.4 μL was charged into the test tube.

The dNTP purchased from Roche was used as the dNTP contained in the reagent 11, and only 0.54 μL was charged into the test tube.

A forward primer and a reverse primer were prepared as the primers contained in the reagent 11. A forward primer for mycoplasma, and a reverse primer for mycoplasma were charged into the test tube in an amount of 0.8 μL each. A forward primer for Escherichia coli, and a reverse primer for Escherichia coli were charged into the test tube in an amount of 0.8 μL each.

The fluorescently labeled probe contained in the reagent 11 is a fluorescently labeled probe for mycoplasma containing the MGB molecule P4. The fluorescently labeled probe was purchased from Life Technologies, and only 0.6 μL was charged into the test tube.

The buffer contained in the reagent 11 had a composition including 25 mM of MgCl₂, 250 mM of Tris-HCl (pH 9.0), and 125 mM of KCl, and only 2.0 μL was charged into the test tube.

The fluorescently labeled probe for mycoplasma contained in the reagent 11 was charged into the test tube in an amount of 0.6 μL.

The forward primers and the reverse primers for mycoplasma and Escherichia coli, and the fluorescently labeled probe for mycoplasma had the sequences shown in Table 1 below.

TABLE 1

| SEQ ID NO: 1 | Forward primer for mycoplasma | 5′ ATCCAGGTACGGGTGAAGACAC 3′ |
|---|---|---|
| SEQ ID NO: 2 | Reverse primer for mycoplasma | 5′ CGCATCAACAAGTCCTAGCGAAC 3′ |
| SEQ ID NO: 3 | Forward primer for Escherichia coli | 5′ AGGCCTTCGGGTTGTAAAGT 3′ |
| SEQ ID NO: 4 | Reverse primer for Escherichia coli | 5′ GTTAGCCGGTGCTTCTTCTG 3′ |
| SEQ ID NO: 5 | Fluorescently labeled probe for | 5′ FAM-CGGGACGGAAAGACC-NFQ-MGB |
| | mycoplasma | 3′ |

In Table 1, “FAM”, which stands for fluorescein aminohexyl, is a type of reporter fluorescent dye. In Table 1, “NFQ”, which stands for non-fluorescent quencher, is a type of quencher fluorescent dye.

The template nucleic acid solution was introduced into the container 10. After the liquid droplet 20 was formed inside the container 10, the container 10 was installed in the insertion hole 64 of the heating unit 65 of the nucleic acid amplifier 50, and the thermal cycle process and the amplification analysis process were performed.

The thermal cycle process was performed in 50 cycles, with 4 seconds of denaturation reaction period, and 6 seconds of synthesis reaction period. The first heating unit 65B and the second heating unit 65C had the temperature settings of 100° C. and 64° C., respectively.

### Comparative Example 1

A template nucleic acid solution was prepared under the same conditions used in Example 1, except that a fluorescently labeled probe different from that used in Example 1 was used.

The fluorescently labeled probe of Comparative Example 1 is a fluorescently labeled probe for mycoplasma containing no MGB molecule P4. The sequence of the fluorescently labeled probe for mycoplasma is shown in Table 2 below.

TABLE 2

| | | |
|---|---|---|
| SEQ ID NO: 6 | Fluorescently labeled probe for | 5′ FAM-CGGGACGGAAAGACC-BHQ1 3′ |
| | mycoplasma | |

In Table 2, “BHQ”, which stands for black hole quencher, is a type of quencher fluorescent dye.

The template nucleic acid solution was introduced into the container 10. After the liquid droplet 20 was formed inside the container 10, the container 10 was installed in the insertion hole 64 of the heating unit 65 of the nucleic acid amplifier 50, and the thermal cycle process and the amplification analysis process were performed under the same conditions used in Example 1.

### Comparison Between Example 1 and Comparative Example 1

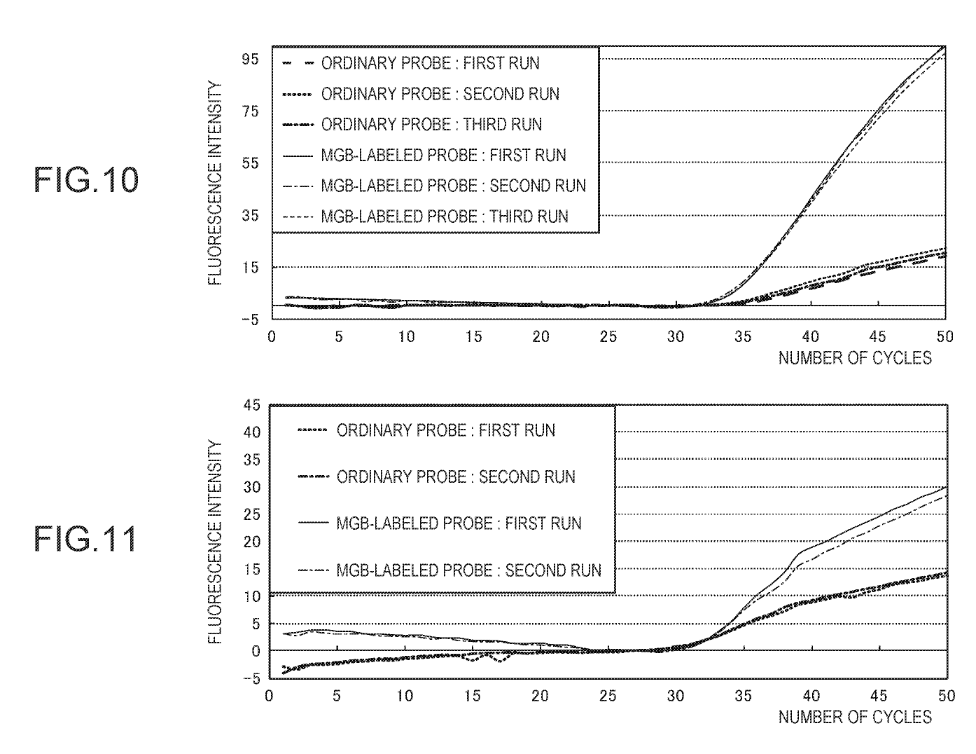

FIG. 10 shows amplification curves for Example 1 and Comparative Example 1. As can be seen in FIG. 10, with the fluorescently labeled probe containing the MGB molecule P4, it was possible to reduce an amplification efficiency drop even when the synthesis reaction period was much shorter than the commonly adopted synthesis reaction period. There was also synergy in that the amplification curve rose a few cycles earlier. That is, it was possible to improve both the detection sensitivity and the specificity of the probe. The observed effect indicates that a plurality of primer sets can be used at the same time, as shown in FIG. 10.

### Example 2

A template nucleic acid solution was prepared under the same conditions used in Example 1, except that a template nucleic acid, primers, and a probe different from those used in Example 1 were used.

The pertussis genomic DNA (MBC008, 200 copies) available from Vircell was used as the template nucleic acid of Example 2.

The primers for pertussis, and the fluorescently labeled probe for pertussis had the sequences shown in Table 3 below.

TABLE 3

| | | |
|---|---|---|
| SEQ ID NO: 7 | Forward primer for pertussis | 5′ ATCCAGGTACGGGTGAAGACAC 3′ |
| SEQ ID NO: 8 | Reverse primer for pertussis | 5′ CGCATCAACAAGTCCTAGCGAAC 3′ |
| SEQ ID NO: 9 | Fluorescently labeled probe | 5′ AM-AATGGCAAGGCCGAACGCTTCA-NFQ- |
| | for pertussis | MGB 3′ |

The template nucleic acid solution was introduced into the container 10. After the liquid droplet 20 was formed inside the container 10, the container 10 was installed in the insertion hole 64 of the heating unit 65 of the nucleic acid amplifier 50, and the thermal cycle process and the amplification analysis process were performed under the same conditions used in Example 1.

### Comparative Example 2

A template nucleic acid solution was prepared under the same conditions used in Example 2, except that a fluorescently labeled probe different from that used in Example 2 was used.

The fluorescently labeled probe of Comparative Example 2 is a fluorescently labeled probe for pertussis containing no MGB molecule P4. The sequence of the fluorescently labeled probe for pertussis is shown in Table 4 below.

TABLE 4

| | | |
|---|---|---|
| SEQ ID NO: 10 | Fluorescently labeled probe | 5′ FAM-AATGGCAAGGCCGAACGCTTCA-BHQ1 3′ |
| | for pertussis | |

The template nucleic acid solution was introduced into the container 10. After the liquid droplet 20 was formed inside the container 10, the container 10 was installed in the insertion hole 64 of the heating unit 65 of the nucleic acid amplifier 50, and the thermal cycle process and the amplification analysis process were performed under the same conditions used in Example 2.

### Comparison Between Example 2 and Comparative Example 2

FIG. 11 shows amplification curves for Example 2 and Comparative Example 2. As can be seen in FIG. 11, with the fluorescently labeled probe containing the MGB molecule P4, it was possible to reduce an amplification efficiency drop even when the synthesis reaction period was much shorter than the commonly adopted synthesis reaction period. The observed effect indicates that a plurality of primer sets can be used at the same time, as shown in FIG. 11.

### Example 3

A template nucleic acid solution was prepared under the same conditions used in Example 1, except that a template nucleic acid, primers, and a probe different from the template nucleic acid, the positive control, the primers, and the probe used in Example 1 were used.

The adenovirus genomic DNA (MBC001, 100 copies) available from Vircell was used as the template nucleic acid of Example 3. The genomic DNA of culture-derived Bacillus subtilis was used as a positive control in Example 3.

The forward primers and the reverse primers for adenovirus and Bacillus subtilis, and the fluorescently labeled probe for adenovirus had the sequences shown in Table 5 below.

TABLE 5

| | | |
|---|---|---|
| SEQ ID NO: 11 | Forward primer for adenovirus | 5′ GACATGACTTTCGAGGTCGATCCCATGGA 3′ |
| SEQ ID NO: 12 | Reverse primer for adenovirus | 5′ CCGGCTGAGAAGGGTGTGCGCAGGTA 3′ |
| SEQ ID NO: 13 | Forward primer for Bacillus subtilis | 5′ TGAAGGTGACGCTGAGTACG 3′ |
| SEQ ID NO: 14 | Reverse primer for Bacillus subtilis | 5′ TTTTTCAGTGTCGCGTTCTG 3′ |
| SEQ ID NO: 15 | Fluorescently labeled probe for | 5 FAM-GAGTGCACCAGCCACACCGC-NFQ- |
| | adenovirus | MGB 3′ |

The template nucleic acid solution was introduced into the container 10. After the liquid droplet 20 was formed inside the container 10, the container 10 was installed in the insertion hole 64 of the heating unit 65 of the nucleic acid amplifier 50, and the thermal cycle process and the amplification analysis process were performed.

The thermal cycle process was performed in 50 cycles, with 4 seconds of denaturation reaction period, and 6 seconds of synthesis reaction period. The first heating unit 65B and the second heating unit 65C had the temperature settings of 100° C. and 58° C., respectively.

### Comparative Example 3

A template nucleic acid solution was prepared under the same conditions used in Example 3, except that a fluorescently labeled probe different from that used in Example 3 was used.

The fluorescently labeled probe of Comparative Example 3 is a fluorescently labeled probe for adenovirus containing no MGB molecule P4. The sequence of the fluorescently labeled probe for adenovirus is shown in Table 6 below.

TABLE 6

| | | |
|---|---|---|
| SEQ ID NO: 16 | Fluorescentl labeled probe for | 5′ FAM-GAGTGCACCAGCCACACCGC-BHQ1 3′ |
| | adenovirus | |

The template nucleic acid solution was introduced into the container 10. After the liquid droplet 20 was formed inside the container 10, the container 10 was installed in the insertion hole 64 of the heating unit 65 of the nucleic acid amplifier 50, and the thermal cycle process and the amplification analysis process were performed under the same conditions used in Example 3.

### Comparison Between Example 3 and Comparative Example 3

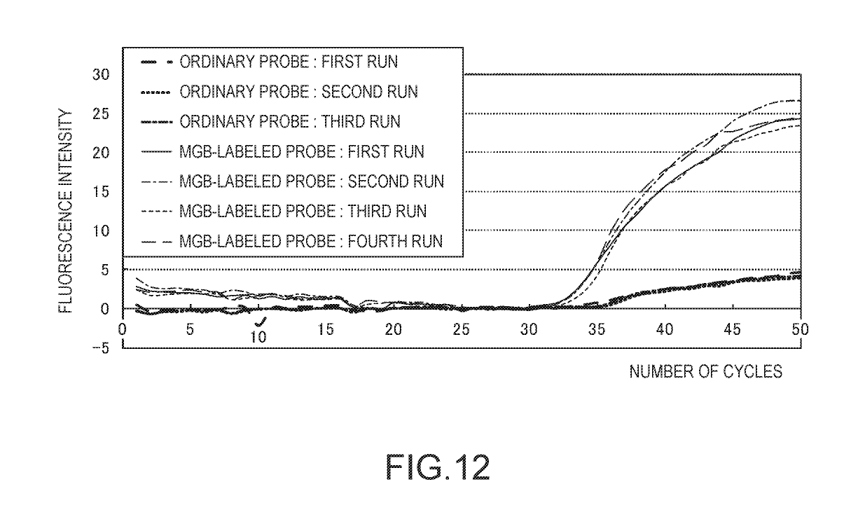

FIG. 12 shows amplification curves for Example 3 and Comparative Example 3. As can be seen in FIG. 12, with the fluorescently labeled probe containing the MGB molecule P4, it was possible to reduce an amplification efficiency drop even when the synthesis reaction period was much shorter than the commonly adopted synthesis reaction period. There was also synergy in that the amplification curve rose a few cycles earlier. That is, it was possible to improve both the detection sensitivity and the specificity of the probe. The observed effect indicates that a plurality of primer sets can be used at the same time, as shown in FIG. 12.

The entire disclosure of Japanese Patent Application No. 2015-106175, filed May 26, 2015 is expressly incorporated by reference herein.

1. A nucleic acid amplifier comprising:
a mount for installing a container containing a liquid droplet and an oil, the liquid droplet containing a template nucleic acid and a reagent used for amplification of a target nucleic acid in the template nucleic acid, the oil having a specific gravity different from a specific gravity of the liquid droplet and being in a separate phase from the liquid droplet;
a first heater that sets a first region of the container installed in the mount to a denaturation temperature of the target nucleic acid, and a second heater that sets a second region of the container different from the first region to a synthesis temperature of the target nucleic acid;
a moving mechanism that moves the liquid droplet from the first region to the second region, and from the second region to the first region; and
a control unit that controls the moving mechanism so as to repeat a denaturation phase of retaining the liquid droplet in the first region, and a synthesis phase of retaining the liquid droplet in the second region in multiple cycles,
the reagent containing a DNA polymerase, primers, a dNTP, and a fluorescently labeled probe,
the fluorescently labeled probe containing a minor groove binder molecule.

2. The nucleic acid amplifier according to claim 1, wherein the period of the synthesis phase of retaining the liquid droplet in the second region is less than 20 seconds.

3. The nucleic acid amplifier according to claim 1, wherein the period of the synthesis phase of retaining the liquid droplet in the second region is 6 seconds or less.

4. A cartridge for nucleic acid amplification, comprising:
a container to which a liquid droplet of a solution containing a template nucleic acid is introduced, and that has a channel for moving the liquid droplet; and
a reagent contained in the container, and that is used for amplification of a target nucleic acid in the template nucleic acid,
the reagent containing a DNA polymerase, primers, a dNTP, and a fluorescently labeled probe,
the fluorescently labeled probe containing a minor groove binder molecule.

5. The cartridge for nucleic acid amplification according to claim 4, wherein the liquid droplet has a volume of 0.2 μL or more and 2 μL or less.

6. The cartridge for nucleic acid amplification according to claim 4, wherein the reagent is freeze dried, and fixed to an inner wall of the container.

7. The cartridge for nucleic acid amplification according to claim 4, comprising an oil contained in the container, the oil having a specific gravity different from a specific gravity of the reaction solution and being in a separate phase from the reaction solution.

8. The cartridge for nucleic acid amplification according to claim 4, wherein the target nucleic acid has a synthesis reaction time of less than 20 seconds.

9. The cartridge for nucleic acid amplification according to claim 4, wherein the target nucleic acid has a synthesis reaction time of 6 seconds or less.

10. The cartridge for nucleic acid amplification according to claim 4, wherein the primers include a forward primer and a reverse primer,
the forward primer having the sequence 5′ ATCCAGGTACGGGTGAAGACAC 3′,
the reverse primer having the sequence 5′ CGCATCAACAAGTCCTAGCGAAC 3′,
the fluorescently labeled probe having the sequence 5′ FAM-CGGGACGGAAAGACC-NFQ-MGB 3′.

11. The cartridge for nucleic acid amplification according to claim 4, wherein the primers include a forward primer and a reverse primer,
the forward primer having the sequence 5′ ATCCAGGTACGGGTGAAGACAC 3′,
the reverse primer having the sequence 5′ CGCATCAACAAGTCCTAGCGAAC 3′,
the fluorescently labeled probe having the sequence 5′ FAM-AATGGCAAGGCCGAACGCTTCA-NFQ-MGB 3′.

12. The cartridge for nucleic acid amplification according to claim 4, wherein the primers include a forward primer and a reverse primer,
the forward primer having the sequence 5′ GACATGACTTTCGAGGTCGATCCCATGGA 3′,
the reverse primer having the sequence 5′ CCGGCTGAGAAGGGTGTGCGCAGGTA 3′,
the fluorescently labeled probe having the sequence 5′ FAM-GAGTGCACCAGCCACACCGC-NFQ-MGB 3′.

13. A nucleic acid amplification method comprising:
a temperature adjusting step of setting a target nucleic acid denaturation temperature for a first region of a container containing a liquid droplet containing a template nucleic acid and a reagent used for amplification of a target nucleic acid in the template nucleic acid, and setting a target nucleic acid synthesis temperature for a second region different from the first region; and
an amplification step of repeating a denaturation phase and a synthesis phase in multiple cycles, the denaturation phase being a phase in which the liquid droplet is moved and retained in the first region, and the synthesis phase being a phase in which the liquid droplet is moved and retained in the second region,
the reagent containing a DNA polymerase, primers, a dNTP, and a fluorescently labeled probe,
the fluorescently labeled probe containing a minor groove binder molecule.
